(19) United States
(12) Patent Application Publication
(10) Pub. No.: US 2025/0194923 A1
(43) Pub. Date: Jun. 19, 2025

(54) EXAMINATION SYSTEM

(71) Applicant: SONY GROUP CORPORATION, TOKYO (JP)

(72) Inventors: MOTOAKI KOBAYASHI, TOKYO (JP); TOMOYUKI OOTSUKI, TOKYO (JP); YUGO KATSUKI, TOKYO (JP)

(21) Appl. No.: 18/846,495

(22) PCT Filed: Feb. 24, 2023
(86) PCT No.: PCT/JP2023/006670

§ 371 Date: Sep. 12, 2024

(30) Foreign Application Priority Data
Mar. 22, 2022 (JP) 2022-045372

(51) Int. Cl. A61B 3/14; A61B 3/117; A61B 3/135

(52) U.S. Cl. CPC A61B 3/145; A61B 3/117; A61B 3/1173; A61B 3/1176; A61B 3/135

(57) ABSTRACT

An examination system (100) includes a measurement unit (2) that is movable to change an angle relative to an eyeball (200) of a subject and rotatably moved around an eye axis of the eyeball (200) as a rotation axis, an illumination unit (3) that is mounted on the measurement unit (2) to irradiate the eyeball (200), a first camera unit (4L) that is mounted on the measurement unit (2) to image the eyeball (200), and a second camera unit (4R) that is mounted on the measurement unit (2) to image the eyeball (200), in which the first camera unit (4L) and the second camera unit (4R) are independently movable on the measurement unit (2) to change angles relative to the eyeball (200).

### FIELD

The present disclosure relates to an examination system.

### BACKGROUND

For example, Patent Literature 1 discloses a device that images an eyeball microscopically observed.

### CITATION LIST

### Patent Literature

Patent Literature 1: JP 2016-159073 A

### SUMMARY

### Technical Problem

The device of Patent Literature 1 requires manual operations by an ophthalmologist. In addition, the device of Patent Literature 1 is not configured to image the eyeball in a mode suitable for various examinations.

One aspect of the present disclosure is to enable imaging of an eyeball in a mode suitable for examinations.

### Solution to Problem

An examination system according to one aspect of the present disclosure includes: a measurement unit that is movable to change an angle relative to an eyeball of a subject and rotatably moved around an eye axis of the eyeball as a rotation axis; an illumination unit that is mounted on the measurement unit to irradiate the eyeball; a first camera unit that is mounted on the measurement unit to image the eyeball; and a second camera unit that is mounted on the measurement unit to image the eyeball, wherein the first camera unit and the second camera unit are independently movable on the measurement unit to change angles relative to the eyeball.

### BRIEF DESCRIPTION OF DRAWINGS

FIG. 1 is a diagram illustrating an exemplary schematic configuration of an examination system according to an embodiment.

FIG. 2 is a diagram schematically illustrating an example of movement of each unit.

FIG. 3 is a diagram illustrating an example of gonioscopy.

FIG. 4 is a diagram illustrating an example of corneal examination.

FIG. 5 is a diagram illustrating an example of crystalline lens examination.

FIG. 6 is a diagram illustrating an example of the crystalline lens examination.

FIG. 7 is a diagram illustrating an example of the crystalline lens examination.

FIG. 8 is a diagram illustrating an example of examination of Zinn's zonule.

FIG. 9 is a diagram illustrating an example of corneal endothelial cell examination.

FIG. 10 is a diagram schematically illustrating an example of imaging using a reflector unit.

FIG. 11 illustrates diagrams of exemplary shapes of the reflector unit.

FIG. 12 is a diagram illustrating an example of presentation of a fixation target.

FIG. 13 is a diagram illustrating an example of presentation of the fixation target.

FIG. 14 is a diagram illustrating an example of presentation of the fixation target.

FIG. 15 is a flowchart illustrating an example of image processing.

FIG. 16 is an exemplary flowchart illustrating Step S2 of FIG. 15 in detail.

FIG. 17 is an exemplary flowchart illustrating Step S3 of FIG. 15 in detail.

FIG. 18 is an exemplary flowchart illustrating Step S4 of FIG. 15 in detail.

FIG. 19 is an exemplary flowchart illustrating Step S5 of FIG. 15 in detail.

FIG. 20 is an exemplary flowchart illustrating Step S6 of FIG. 15 in detail.

FIG. 21 is a diagram illustrating an exemplary hardware configuration of a control processing device 20.

### DESCRIPTION OF EMBODIMENTS

Embodiments of the present disclosure will be described in detail below with reference to the drawings. Note that in the following embodiments, the same elements are denoted by the same reference numerals, and repetitive description thereof will be omitted.

Furthermore, the present disclosure will be described in the order of items shown below.
0. Introduction
1. Embodiments
1.1. Anterior segment examination
1.2. Gonioscopy
1.3. Corneal examination
1.4. Crystalline lens examination
1.5. Examination of Zinn's zonule
1.6. Corneal endothelial cell examination
1.7. Reflector unit
1.8. Fixation target
1.9. Image processing
2. Modifications
3. Exemplary hardware configuration
4. Exemplary effects

### 0. Introduction

An ophthalmologist diagnoses the condition of an eye of a patient by using a slit lamp upon examination. The slit lamp was invented more than 100 years ago, and is an analog ophthalmic diagnostic device that has been widely spread. At present, various digitized examination devices have been developed for diagnosis, but are each a device that performs single-function examination. In a workflow of diagnosis, preliminary examination is performed by using various examination devices in an examination room before diagnosis by an ophthalmologist, and then the ophthalmologist makes a diagnosis by using the slit lamp in a consultation room. Then, depending on a suspected disease, a necessary examination is performed again in the examination room by using another examination device. Finally, returning to the consultation room, the ophthalmologist makes a diagnosis again. In such a flow, diagnoses are made by many ophthalmologists.

The workflow of preliminary examination, diagnosis, another examination, and another diagnosis as described above requires a lot of time for diagnosis. In addition, examinations performed by a plurality of examination devices require a space for the examination room, and preparation of the devices is expensive. If it is possible for a single device to complete necessary examinations at a time in advance and further to present results of the examinations upon preliminary examination, diagnosis can be made at one time, enabling a significant high-speed workflow.

Existing slit lamps generally have no imaging function for image data, and only monocular imaging devices having low resolutions are available even when the imaging devices have imaging functions. Therefore, there is no device that is configured to acquire image quality at a level that can be used for diagnosis by the ophthalmologist and that can be acquire three-dimensional data (3D data), in the examination room in advance. In the technique of Patent Literature 1, the structure itself of the slit lamp is not different from that of a conventional slit lamp, and thus the slit lamp is provided as a device that is manually operated and used by the ophthalmologist. There is a problem that the technique of Patent Literature 1 is not suitably used to acquire data in the examination room in advance. In addition, there is a case where it is desired to acquire data about three-dimensional shapes of a corneal cell, Zinn's zonule, and a cornea, and a three-dimensional shape and the like of a crystalline lens, for use in diagnosis. However, these data cannot be acquired, and therefore, it is also necessary to use different examination devices for diagnoses, if required.

For example, at least some of the problems described above are addressed by a disclosed technology. For example, an illumination unit that illuminates an eye to be examined under a desired condition, and two imaging units are used. According to the examination content, the illumination method of the illumination unit, the positions of the illumination unit and the imaging unit, and the like are adjusted to optimum conditions, and imaging is performed. At the same time, analysis is performed, with presentation or the like of information used for diagnosis.

### 1. Embodiments

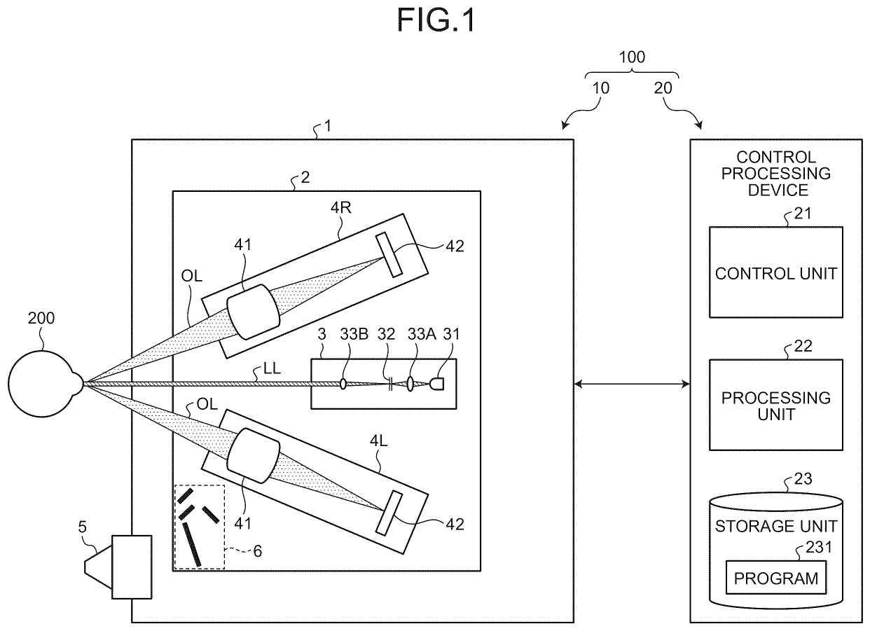

FIG. 1 is a diagram illustrating an exemplary schematic configuration of an examination system according to an embodiment. The examination system 100 is used for eye examination, eye diagnosis, and the like. An eyeball portion of an eye as a target to be examined is illustrated as an eyeball 200. The examination system 100 includes an imaging device 10 and a control processing device 20. FIG. 1 schematically illustrates an arrangement of the eyeball 200 and the imaging device 10 as viewed from above (in plan view).

The imaging device 10 images the eyeball 200. The imaging device 10 includes a base unit 1, a measurement unit 2, an illumination unit 3, a camera unit 4L, a camera unit 4R, a gonioscope unit 5, and a reflective optical system unit 6. In this example, the measurement unit 2 and the gonioscope unit 5 are mounted on the base unit 1. The illumination unit 3, the camera unit 4L, the camera unit 4R, and the reflective optical system unit 6 are mounted on the measurement unit 2.

The illumination unit 3 irradiates the eyeball 200 with illumination light LL to illuminate the eyeball 200. An example of the illumination light LL includes slit lamp light, and the illumination light LL being the slip lamp light has a slit shape (rectangular shape) in a plane orthogonal to the optical path. The illumination light LL may be light other than the slit lamp light and may have a shape other than the slit shape. Examples of other shapes include a random dot pattern shape, a grid pattern shape, and the like. In the following description, it is assumed that the illumination light LL is the slit lamp light having the slit shape.

The illumination unit 3 includes a light source 31, an optical system 33A, a slit 32, and an optical system 33B. The light source 31 includes, for example, a light emitting diode (LED), a laser diode (LD), or the like. The light source 31 may have a plurality of light sources that can be switchably used. Examples of the light source include a white light source and an infrared light source.

The illumination light LL output by the light source 31 is condensed by the optical system 33A, then narrowed into a slit shape by the slit 32, collimated into parallel light by the optical system 33B, and output. The slit 32 has a controllable width to adjust the width of the illumination light LL (the width of the slit lamp light).

The camera unit 4L and the camera unit 4R image the eyeball 200, more specifically, a portion (e.g., a cross-section) of the eyeball 200 illuminated with the illumination light LL from the illumination unit 3. Light from the eyeball 200 toward the camera unit 4L and the camera unit 4R is illustrated as observation light OL. The camera unit 4L and the camera unit 4R each receive the observation light OL to image the eyeball 200.

The camera unit 4L is a first camera unit (left camera unit) positioned on the left side from the camera unit 4R when the eyeball 200 is viewed from the measurement unit 2. The camera unit 4R is a second camera unit (right camera unit) positioned on the right side from the camera unit 4L. The camera unit 4L and the camera unit 4R may have the same configuration.

Each of the camera unit 4L and the camera unit 4R includes an objective zoom optical system 41 and an image sensor 42. The illustrated objective zoom optical system 41 is an objective lens. The objective zoom optical system may be understood to include an optical system having variable magnification. The image sensor 42 images the eyeball 200 observed (e.g., magnifying observation) through the objective zoom optical system 41.

The objective zoom optical system 41 and the image sensor 42 of the camera unit 4L and the objective zoom optical system 41 and the image sensor 42 of the camera unit 4R are configured to be controlled independently. The magnification of the camera unit 4L and the magnification of the camera unit 4R are also configured to be set independently. The camera unit 4L and the camera unit 4R configured in this way function as a Greenough type stereo camera that images the eyeball 200 at different positions, angles, magnifications, and the like.

Data about an image of the eyeball 200 captured by each of the camera unit 4L and the camera unit 4R is transmitted from the imaging device 10 to the control processing device 20. Note that “image” and “imaging” may be understood to include “video” and “image capturing”. The image and the imaging may be appropriately replaced with the video and the image capturing, respectively, as long as there is no contradiction. In addition, hereinafter, the image data is also simply referred to as an image or the like.

The gonioscope unit 5 and the reflective optical system unit 6 are used to image an angle region of the eyeball 200. The gonioscope unit 5 includes a gonioscope. The reflective optical system unit 6 includes an optical element that reflects the illumination light LL and the observation light OL.

A patient support, which is not illustrated, or the like may be provided. The patient support may include a chin rest that stably supports a jaw of the patient placed thereon, a forehead rest that stabilizes the forehead placed thereon, a fixation target for aligning the line of sight in a certain direction to stabilize the position of the eyeball 200, and the like. The chin rest, the forehead rest, the fixation target, and the like may be unitized and incorporated (e.g., integrated) into the imaging device 10. Using the patient support makes it possible to stabilize the position of the eyeball 200 with respect to the imaging device 10.

The units described above included in the imaging device 10 are movable independently. The movements of the units are independently controlled by, for example, an electric drive mechanism or a device, which is not illustrated.

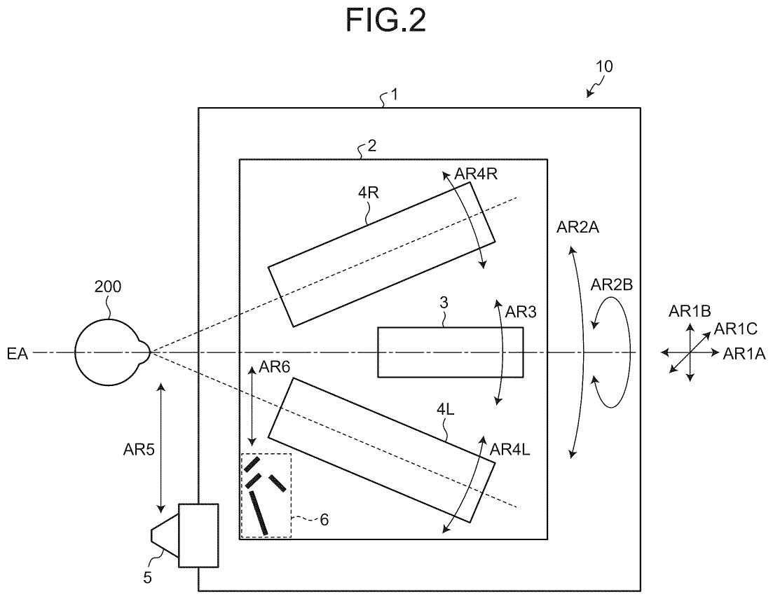

FIG. 2 is a diagram schematically illustrating an example of movement of each of the units. An eye axis of the eyeball 200 is schematically illustrated by a dash-dot-dash line as an eye axis EA.

The movement of the base unit 1 is schematically indicated by an arrow AR1A, an arrow AR1B, and an arrow AR1C. The base unit 1 is movable forward and backward, leftward and rightward, and vertically, relative to the eyeball 200. The respective units on the base unit 1, for example, the illumination unit 3, the camera unit 4L, the camera unit 4R, and the like are allowed to be readily aligned at positions suitable for the examination of the eyeball 200.

The movement of the measurement unit 2 is schematically indicated by arrows AR2A and AR2B. As indicated by the arrow AR2A, the measurement unit 2 is movable, for example, with the eyeball 200 or the vicinity of a front end portion thereof as a rotation center so as to change an angle relative to (the eye axis EA of) the eyeball 200. It can also be said that the movement of the measurement unit 2 indicated by the arrow AR2A is a horizontal swing movement relative to the eyeball 200 in relative position. As indicated by the arrow AR2B, the measurement unit 2 is rotatably moved around the eye axis EA of the eyeball 200 as a rotation axis. In addition, moving the measurement unit 2 makes it possible to move the illumination unit 3, the camera unit 4L, the camera unit 4R, and the reflective optical system unit 6 while fixing the arrangement (relative position) of these units.

For example, the illumination unit 3, the camera unit 4L, and the camera unit 4R are arranged by default as illustrated in FIG. 2. The illumination unit 3 is directed so that a straight line (eye axis EA in this example) extending from the eyeball 200 toward the illumination unit 3 passes through the center of the illumination unit 3. The camera unit 4L and the camera unit 4R are arranged on the opposite sides across the illumination unit 3. As indicated by a broken line in FIG. 2, the camera unit 4L is directed so that a straight line extending from the eyeball 200 toward the camera unit 4L passes through the center of the camera unit 4L. The camera unit 4R is directed so that a straight line extending from the eyeball 200 toward the camera unit 4R passes through the center of the camera unit 4R.

The movement of the illumination unit 3 is schematically indicated by an arrow AR3. The movement of the camera unit 4L is schematically indicated by an arrow AR4L. The movement of the camera unit 4R is schematically indicated by an arrow AR4R. Similarly to the movement of the measurement unit 2 indicated by the arrow AR2A described above, the illumination unit 3, the camera unit 4L, and the camera unit 4R are independently movable on the measurement unit 2 to change the angles relative to the eyeball 200.

On the measurement unit 2, the angles of the illumination unit 3, the camera unit 4L, and the camera unit 4R relative to the eyeball 200 are allowed to be independently controlled. For example, an inward angle between the camera unit 4L and the camera unit 4R is allowed to be changed, or the camera unit 4L and the camera unit 4R is allowed to be moved together while maintaining a positional relationship between the camera units.

The movement of the gonioscope unit 5 is schematically indicated by an arrow AR5. The gonioscope unit 5 is movable between a position (insertion position) located between the eyeball 200 and the measurement unit 2, and another position (retraction position) other than the insertion position, in an insertable manner. More specifically, the insertion position is located at a position between the eyeball 200, and, the illumination unit 3 and at least one of the camera unit 4L and the camera unit 4R. In the example illustrated in FIG. 2, the insertion position is located between the eyeball 200, and, the illumination unit 3 and the camera unit 4L. The insertion position is located at a position on the optical path of the illumination light LL and on the optical path of the observation light OL to the camera unit 4L. The retraction position is located at a position outside of the optical path of the illumination light LL and outside of the optical path of the observation light OL to the camera unit 4L. The retraction position may be located at any position where the movements of the illumination unit 3, the camera unit 4L, and the camera unit 4R are not hindered.

The movement of the reflective optical system unit 6 is schematically indicated by an arrow AR6. The reflective optical system unit 6 is movable between a position between the gonioscope unit 5, and the illumination unit 3 and the camera unit 4L, and the other position, in an insertable manner. These positions of the reflective optical system unit 6 are similar to the insertion position and the retraction position of the gonioscope unit 5 described above, and a repetitive description thereof will be omitted.

Returning to FIG. 1, the control processing device 20 will be described. The control processing device 20 includes a control unit 21, a processing unit 22, and a storage unit 23.

The control unit 21 controls each unit of the imaging device 10 to control imaging of the eyeball 200 by the imaging device 10. For example, the control unit 21 controls the movement of the base unit 1. The control unit 21 controls the movement of the measurement unit 2. The control unit 21 controls the movement of the illumination unit 3 and controls illumination by the illumination unit 3. The controlling of illumination includes parameter control. Examples of the parameter include an internal parameter such as the shape (slit width etc.), wavelength, or illuminance of the illumination light LL, and an external parameter such as the position, orientation, or attitude of the illumination unit 3.

The control unit 21 controls the movement of the camera unit 4L and controls imaging by the camera unit 4L. The controlling of imaging includes parameter control. Examples of the parameter include an internal parameter such as the focal length or the focal position (including the magnification of the objective zoom optical system 41) of the camera unit 4L, and an external parameter such as the position, orientation, or attitude of the camera unit 4L. Similarly, the control unit 21 controls the movement of the camera unit 4R and controls imaging by the camera unit 4R.

The processing unit 22 processes the image of the eyeball 200 captured by the imaging device 10. An image related to the examination of the eyeball 200, for example, an image or the like that can be provided for diagnosis of the eyeball 200 is generated. The eyeball 200 may be diagnosed on the basis of the image.

The storage unit 23 stores information used in the control processing device 20. A program 231 is exemplified as the information stored in the storage unit 23. The program 231 is a control processing program (software) for causing a computer to function as the control processing device 20.

According to the examination system 100 described above, independent control of the movement of each unit makes it possible to image the eyeball 200 in various modes suitable for various examinations. Use of the examination system 100 eliminates the need for an operation of the slit lamp by an ophthalmologist, and a person other than the ophthalmologist can be an examiner. For example, completion of various examinations with the examination system 100 before the diagnosis by the ophthalmologist enables a significant high-speed workflow of diagnosis.

Some examples will be described. Unless otherwise specified, movement or the like of each unit of the imaging device 10 is controlled by the control unit 21 of the control processing device 20. Each of the camera unit 4L and the camera unit 4R has a magnification that is set to a magnification suitable for imaging a target to be imaged. In addition, unless otherwise specified, the magnification of each of the camera unit 4L and the camera unit 4R is set to the same magnification.

### 1.1. Anterior Segment Examination

For example, an anterior segment examination is performed first. According to instructions from the examiner, a subject (patient) places his/her chin on the chin rest, puts his/her forehead against the forehead rest, and then looks at the fixation target. In this state, the base unit 1 moves so that the center of the eyeball 200 of the subject is aligned with the center of the illumination unit 3 (so that the eye axis EA of the eyeball 200 passes through the center of the illumination unit 3). In an example, the position of the base unit 1 is adjusted to a position at which an image generated by light projected from the fixation target and reflected from the cornea of the eyeball 200 of the subject coincides with a center position of the illumination light LL of the illumination unit 3 and the best sharpness is achieved. Note that the movement of the base unit 1 may be manually controlled by the examiner.

The illumination unit 3, the camera unit 4R, and the camera unit 4L on the measurement unit 2 are arranged to have a positional relationship illustrated in FIGS. 1 and 2 described above. The camera unit 4L and the camera unit 4R are arranged on the opposite sides across the illumination unit 3.

In the above state, as indicated by the arrow AR2A in FIG. 2, the measurement unit 2 moves to change the angle relative to the eyeball 200. Along with this movement, each of the camera unit 4L and the camera unit 4R images an anterior segment of the eyeball 200. Imaging is performed with the movement of the measurement unit 2 as a scan operation. Therefore, an image corresponding to the anterior segment of the eyeball 200 observed by the ophthalmologist with the slit lamp is obtained.

The obtained image is stored in the storage unit 23 of the control processing device 20 and processed by the processing unit 22. For example, an image that can be used for diagnosis of the anterior segment of the eyeball 200 is generated and displayed on a monitor which is not illustrated or the like. The image may be displayed in real time.

The processing unit 22 may perform diagnosis based on the image obtained in the anterior segment examination described above. Various known algorithms (including machine learning models) may be used. The processing unit 22 determines necessity of further examination of a region of the eyeball 200, and presents information about items or the like of the necessary examination to the examiner. Further examination of the region of the eyeball 200 is performed by using the examination system 100. Examples of the region of the eyeball 200 that can be the target to be examined include an angle region, a cornea, a crystalline lens, and the like, and for example, examinations as described later are performed.

### 1.2. Gonioscopy

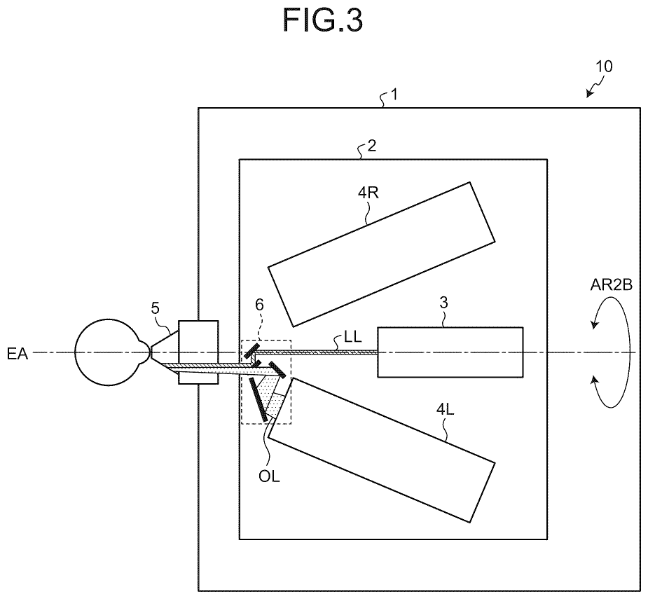

FIG. 3 is a diagram illustrating an example of gonioscopy. The eyeball 200 is imaged so that the state of the angle region of the eyeball 200 can be grasped entirely circumferentially. The gonioscope unit 5 described above is used, and the reflective optical system unit 6 is also used, in this example. The gonioscope unit 5 and the reflective optical system unit 6 are each arranged at a position between the eyeball 200, and the illumination unit 3 and the camera unit 4L. The illumination unit 3 is arranged at a position where the angle region of the eyeball 200 is irradiated through the reflective optical system unit 6 and the gonioscope unit 5. The camera unit 4L is arranged at a position where the angle region of the eyeball 200 is imaged via the gonioscope unit 5 and the reflective optical system unit 6.

In the example illustrated in FIG. 3, the optical element (mirror or the like) in the reflective optical system unit 6 reflects the illumination light LL from the illumination unit 3, toward the gonioscope unit 5. In the gonioscope unit 5, for example, reflectors are arranged circumferentially at regular intervals and with equal pitch. Each of the reflectors in the gonioscope unit 5 reflects the illumination light LL from the reflective optical system unit 6 toward the angle region of the eyeball 200. The angle region of the eyeball 200 is illuminated with the illumination light LL.

In addition, each reflector in the gonioscope unit 5 reflects the observation light OL from the angle region of the eyeball 200 toward the reflective optical system unit 6. The optical element in the reflective optical system unit 6 reflects the observation light OL from the gonioscope unit 5 toward the camera unit 4L. The angle region of the eyeball 200 is observed by the camera unit 4L.

In the above state, as indicated by the arrow AR2B in FIG. 3, the measurement unit 2 rotationally moves around the eye axis EA of the eyeball 200 as the rotation axis. Along with this movement, the camera unit 4L images the angle region of the eyeball 200. The angle region of the eyeball 200 is imaged in the rotation direction. For example, the measurement unit 2 is rotationally moved until the angle region is imaged entirely circumferentially.

An image obtained is stored in the storage unit 23 of the control processing device 20 and processed by the processing unit 22. For example, an image that can be used for diagnosis of the angle region of the eyeball 200 is generated or displayed on the monitor. Although a display mode is not particularly limited, for example, images corresponding to the respective reflectors in the gonioscope unit 5 may be displayed side by side, or one donut-shaped image in which the images are connected in an annular shape may be displayed.

### 1.3. Corneal Examination

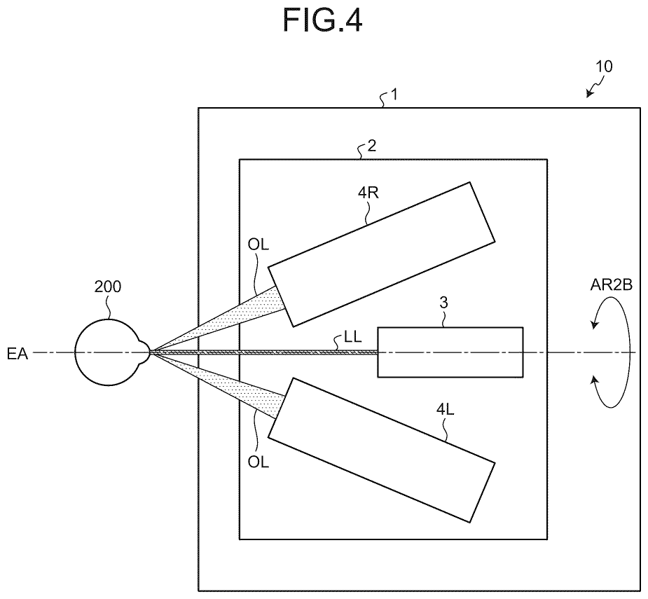

FIG. 4 is a diagram illustrating an example of corneal examination. The gonioscope unit 5 and the reflective optical system unit 6 are not illustrated. The eyeball 200 is imaged so that the state of the cornea of the eyeball 200 can be grasped. The illumination unit 3 is arranged in front of the eyeball 200. The eye axis EA of the eyeball 200 passes through the center of the illumination unit 3. The cornea of the eyeball 200 is irradiated with the illumination light LL from the illumination unit 3.

The camera unit 4L and the camera unit 4R are arranged on the opposite sides across the illumination unit 3 so as to have an angle relative to the eyeball 200. More specifically, the camera unit 4L and the camera unit 4R are arranged axially symmetrically with respect to the eye axis EA of the eyeball 200 so as to have angles of the same magnitude relative to the respective eyeball 200.

In this state, as indicated by the arrow AR2B, the measurement unit 2 rotationally moves around the eye axis EA of the eyeball 200 as the rotation axis. Along with this movement, the camera unit 4L and the camera unit 4R image the cornea of the eyeball 200. The cornea of the eyeball 200 is imaged in the rotation direction. For example, the measurement unit 2 is rotationally moved until an image of the cornea of one cycle is captured.

An image obtained is stored in the storage unit 23 of the control processing device 20 and processed by the processing unit 22. For example, an image that can be used for diagnosis of the cornea of the eyeball 200 is generated or displayed on the monitor. Furthermore, the shape of the cornea of the eyeball 200 is calculated on the basis of the image. For example, data about the shape of the cornea is constructed and the shape is reconstructed.

Note that the shape of the region of the eyeball 200 calculated by the processing unit 22 may be a three-dimensional shape. The shape may be appropriately replaced with the three-dimensional shape as long as there is no contradiction.

### 1.4. Crystalline Lens Examination

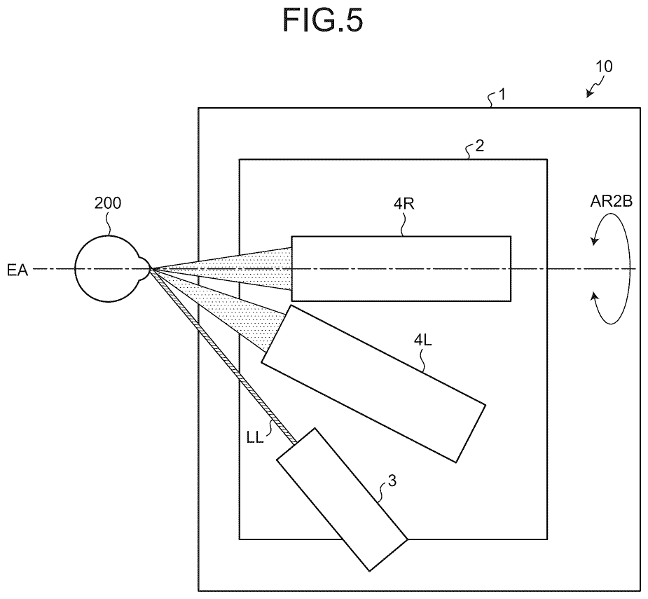

FIGS. 5 to 7 are each a diagram illustrating an example of crystalline lens examination. The eyeball 200 is imaged so that the shape of the crystalline lens of the eyeball 200 or an opacity distribution in the crystalline lens can be grasped.

FIG. 5 illustrates an example of imaging for grasping the shape of the crystalline lens including an equator. The illumination unit 3 is arranged at a position where the illumination unit 3 has an angle relative to the eyeball 200. The crystalline lens of the eyeball 200 is irradiated with the illumination light LL from the illumination unit 3.

The camera unit 4L and the camera unit 4R are each arranged so as to have an inward angle to image the eyeball 200. The lens equator hidden behind an iris when the eyeball 200 is viewed from the front is also imaged. Imaging of the crystalline lens including the equator is useful, for example, for cataract surgery. In the cataract surgery, it is preliminarily performed, for example, to predict the final settling depth of an intraocular lens, grasp the ease of rotation of the intraocular lens in a lens capsule, which causes reduction of a correction effect when the intraocular lens is used to correct astigmatism, and select an appropriate size when the intraocular lens is an accommodation intraocular lens. It is important to grasp the shape of the lens equator of the eyeball 200.

In a state where the camera unit 4L and the camera unit 4R are arranged as described above, the measurement unit 2 rotationally moves around the eye axis EA of the eyeball 200 as the rotation axis, as indicated by the arrow AR2B. At the same time, the camera unit 4L and the camera unit 4R image the crystalline lens of the eyeball 200, more specifically, the lens equator. For example, the measurement unit 2 is rotationally moved until an image of the crystalline lens of one cycle is captured.

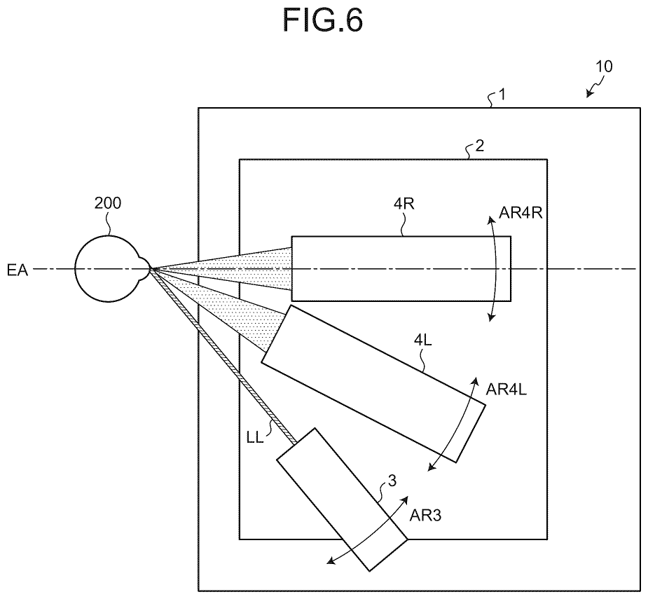

FIG. 6 illustrates an example of imaging for grasping the opacity distribution in the crystalline lens. Similarly to FIG. 5 described above, the illumination unit 3 is arranged at a position where the illumination unit 3 has an angle relative to the eyeball 200. The crystalline lens of the eyeball 200 is irradiated with the illumination light LL from the illumination unit 3.

In this state, the illumination unit 3, the camera unit 4L, and the camera unit 4R are moved together (arrow AR3, arrow AR4L, and arrow AR4R) to change the angles relative to the eyeball 200, while maintaining the positional relationship (angles) therebetween. Along with this movement, the camera unit 4L and the camera unit 4R image the crystalline lens of the eyeball 200. The crystalline lens of the eyeball 200 is imaged in a moving direction from different angles. The crystalline lens of the eyeball 200 is imaged in the moving directions of the illumination unit 3, the camera unit 4L, and the camera unit 4R.

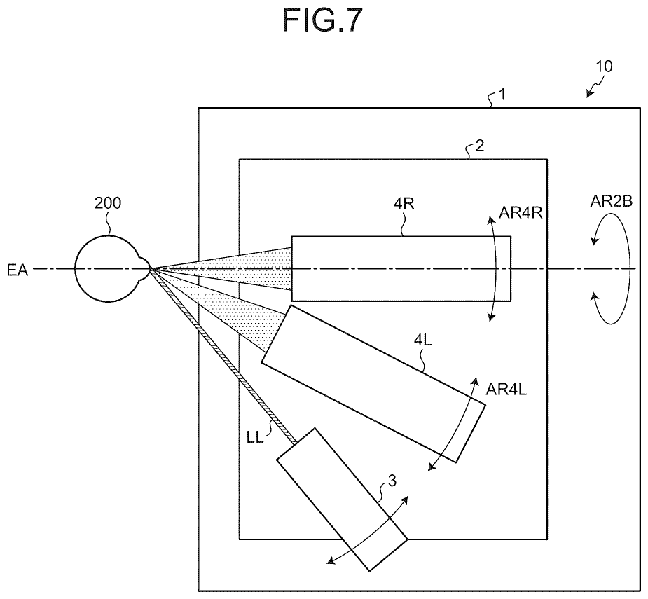

As illustrated in FIG. 7, the rotation of the measurement unit 2 may be further combined. When the movement of the illumination unit 3, the camera unit 4L, and the camera unit 4R, and the imaging by the camera unit 4L and the camera unit 4R are finished, the measurement unit 2 rotationally moves around the eye axis EA of the eyeball 200 as the rotation axis, as indicated by the arrow AR2B. At the same time, the camera unit 4L and the camera unit 4R image the crystalline lens of the eyeball 200. The crystalline lens of the eyeball 200 is imaged in the rotation direction of the measurement unit 2 and in the moving directions of the camera unit 4L and the camera unit 4R. For example, the measurement unit 2 is rotationally moved until an image of the crystalline lens of one cycle is captured.

Note that the positional relationship between the illumination unit 3, the camera unit 4L, and the camera unit 4R is not limited to the examples illustrated in FIGS. 5 to 7 described above. For example, the illumination unit 3 may be positioned between the camera unit 4L and the camera unit 4R.

An image obtained is stored in the storage unit 23 of the control processing device 20 and processed by the processing unit 22. For example, an image that can be used for diagnosis of the crystalline lens of the eyeball 200 is generated or displayed on the monitor. Furthermore, the shape of the crystalline lens of the eyeball 200 is calculated or the opacity distribution in the crystalline lens is calculated, on the basis of the image.

### 1.5. Examination of Zinn's Zonule

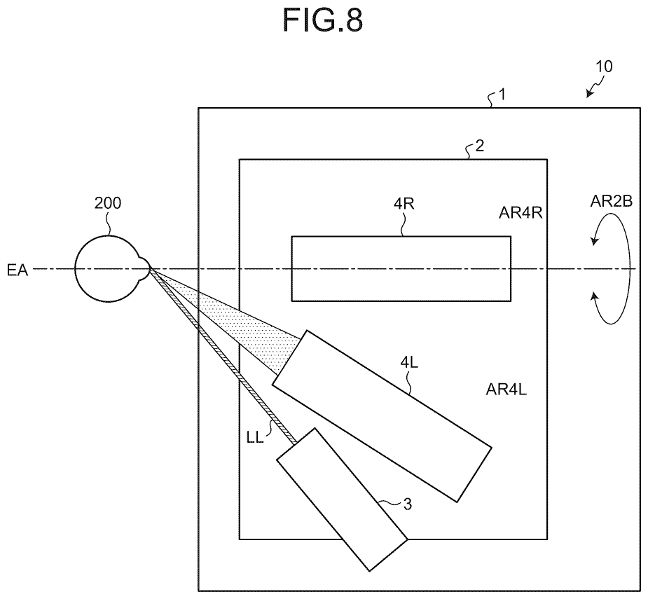

FIG. 8 is a diagram illustrating an example of examination of Zinn's zonule. The eyeball 200 is imaged so that the state of the Zinn's zonule of the eyeball 200 can be grasped entirely circumferentially. The illumination unit 3 is arranged at a position where the illumination unit 3 has an angle relative to the eyeball 200 so as to illuminate the Zinn's zonule of the eyeball 200. The Zinn's zonule of the eyeball 200 is irradiated with the illumination light LL from the illumination unit 3.

At least one of the camera unit 4L and the camera unit 4R is arranged at a position where the at least one of the camera unit 4L and the camera unit 4R has an angle relative to the eyeball 200 so as to observe the Zinn's zonule of the eyeball 200. In this example, the camera unit 4L is arranged at a position where the camera unit 4L has an angle relative to the eyeball 200 to image the Zinn's zonule of the eyeball 200.

In this state, as indicated by the arrow AR2B, the measurement unit 2 rotationally moves around the eye axis EA of the eyeball 200 as the rotation axis. Along with this movement, the camera unit 4L images the Zinn's zonule of the eyeball 200. The Zinn's zonule of the eyeball 200 is imaged in the moving direction. For example, the measurement unit 2 is rotationally moved until an image of the Zinn's zonule of one cycle is captured.

An image obtained is stored in the storage unit 23 of the control processing device 20 and processed by the processing unit 22. For example, an image that can be used for diagnosis (diagnosis for weakness or the like) of the Zinn's zonule of the eyeball 200 is generated or displayed on the monitor.

### 1.6. Corneal Endothelial Cell Examination

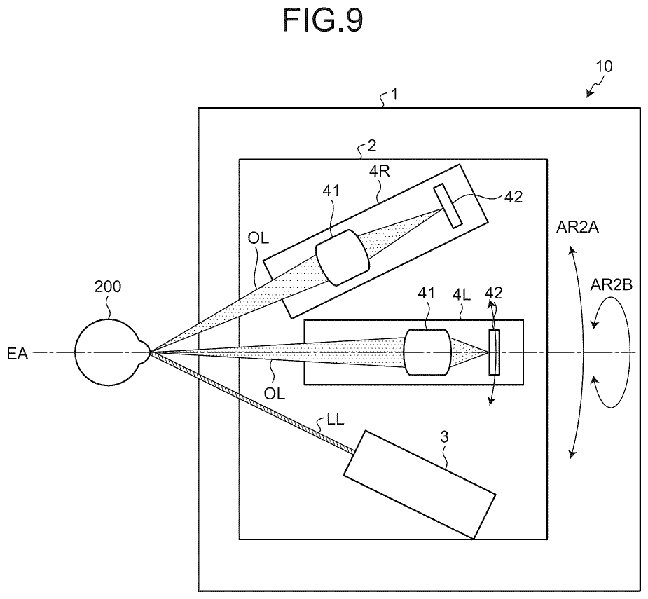

FIG. 9 is a diagram illustrating an example of corneal endothelial cell examination. The eyeball 200 is imaged so that the state of the corneal endothelial cells of the eyeball 200 can be grasped. The illumination unit 3 is arranged at a position where the illumination unit 3 has an angle relative to a line normal to a corneal endothelium as the target to be imaged in the eyeball 200. The corneal endothelial cells of the eyeball 200 are irradiated with the illumination light LL from the illumination unit 3.

The camera unit 4L is arranged in front of the corneal endothelium as the target to be imaged, in the eyeball 200. In a case where the corneal endothelium at the center of the cornea is the target to be imaged, the camera unit 4L is arranged in front of the eyeball 200, as illustrated in FIG. 9. The eye axis EA of the eyeball 200 passes through the center of the camera unit 4L. The objective zoom optical system 41 of the camera unit 4L has a magnification that is set to a magnification (relatively low magnification) at which the entire cornea is observable.

The camera unit 4R is arranged on a side opposite from the illumination unit 3 across the line normal to the corneal endothelium as the target to be imaged, axially symmetrically with respect to the line normal to the corneal endothelium as the target to be imaged. In a case where the corneal endothelium at the center of the cornea is the target to be imaged, the illumination unit 3 and the camera unit 4R are arranged axially symmetrically with respect to the eye axis EA of the eyeball 200, as illustrated in FIG. 9 The objective zoom optical system 41 of the camera unit 4R has a magnification that is set to a magnification (relatively high magnification) at which the corneal endothelial cells are observable.

In the above state, the camera unit 4L captures an image of the entire cornea of the eyeball 200. The illumination light LL (slit lamp light) of the illumination unit 3 is controlled to have a width large enough to illuminate the cornea of the eyeball 200. Then, the position of the base unit 1 is adjusted so that the center of the image captured by the camera unit 4L coincides with the center of the cornea of the eyeball 200.

Next, the camera unit 4R images the corneal endothelial cells of the eyeball 200. The illumination light LL of the illumination unit 3 is controlled to have a width small enough to correspond only to the range of the corneal endothelial cells of the eyeball 200.

Specifically, as indicated by the arrow AR2A, the measurement unit 2 moves to change the angle relative to the eyeball 200. Along with this movement, the camera unit 4R images the corneal endothelial cells of the eyeball 200 along a line passing through the corneal center of the eyeball 200 in a horizontal direction. As indicated by the arrow AR2B, the measurement unit 2 rotationally moves around the eye axis EA of the eyeball 200 as the rotation axis. The line passing through the corneal center is tilted. Along with this movement, the camera unit 4R images the corneal endothelial cells of the eyeball 200.

An image obtained is stored in the storage unit 23 of the control processing device 20 and processed by the processing unit 22. For example, an image that can be used for diagnosis of the corneal endothelial cells is generated or displayed on the monitor. The number of the corneal endothelial cells or the size of each of the corneal endothelial cells may be analyzed, or a result thereof may be displayed on the monitor.

For example, various examinations corresponding to the regions of the eyeball 200 as described above are performed by the examination system 100. Prior to diagnosis by the ophthalmologist, images and the like corresponding to the various examinations can be acquired in advance. The ophthalmologist can make a diagnosis on the basis of the images or the like acquired in advance without observation on the spot using the slit lamp upon diagnosis. Thus, it is possible to perform significantly high-speed workflow of diagnosis as described at the beginning.

### 1.7. Reflector Unit

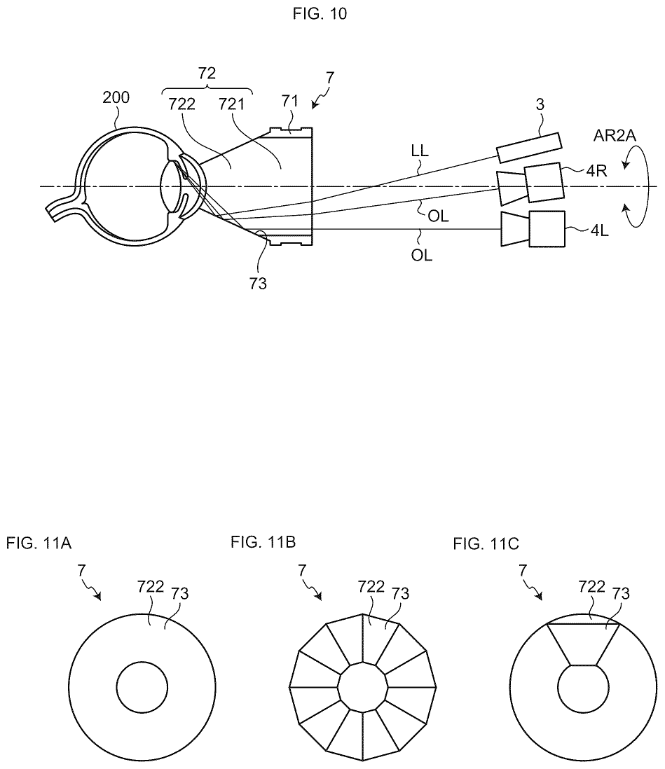

For example, in the crystalline lens examination (FIGS. 5 to 7) and examination of Zinn's zonule (FIG. 8) which have been described above, it is necessary to arrange each of the illumination unit 3 and the camera unit 4 not in front of the eyeball 200 but at a position where each of the illumination unit 3 and the camera unit 4 has a large angle to some extent relative to (the eye axis EA of) the eyeball 200. When the angle is large, there may be a problem that illumination or imaging is hindered by a feature of the face of a subject or the eyelashes of the subject are displayed in the captured image. In order to address such a problem, a reflector unit may be used. The reflector unit will be described with reference to FIGS. 10 and 11.

FIG. 10 is a diagram schematically illustrating an example of imaging using the reflector unit. The exemplified reflector unit 7 has a shape similar to that of the gonioscope unit 5 described above. Similarly to the other units, the reflector unit 7 may also be a component element of the imaging device 10. Similarly to the gonioscope unit 5, the reflector unit 7 is movable between the insertion position and the retraction position in an insertable manner. The reflector unit 7 may be mounted to a place the same as that of the gonioscope unit 5 interchangeably, or may be arranged at a place different from that of the gonioscope unit 5.

The reflector unit 7 includes a frame body 71, a main body 72 that is supported by the frame body 71, and a reflector 73 that is provided at the main body 72. The main body 72 is made of a material that transmits the illumination light LL and the observation light OL.

The main body 72 of the reflector unit 7 includes a base portion 721 that is supported by the frame body 71 and an extending portion 722 that extends from the base portion 721 toward the eyeball 200. The extending portion 722 has a cross-sectional area (e.g., the area of a surface orthogonal to the eye axis EA of the eyeball 200) that decreases with the distance from the base portion 721. The reflector 73 is provided at least at a part of a side surface of the extending portion 722.

In the example illustrated in FIG. 10, the reflector unit 7 is used to image the lens equator of the eyeball 200, and is arranged between the eyeball 200, and the illumination unit 3, the camera unit 4L, and the camera unit 4R. The reflector 73 of the reflector unit 7 reflects the illumination light LL from the illumination unit 3 toward the lens equator of the eyeball 200. In addition, the reflector 73 reflects the observation light OL from the lens equator of the eyeball 200 toward the camera unit 4L and the camera unit 4R.

In a state where the reflector unit 7 is arranged as described above, each of the illumination unit 3, the camera unit 4L, and the camera unit 4R is arranged at a position where the angle relative to the eyeball 200 is smaller than that that of a configuration without the reflector unit 7 (e.g., FIGS. 5 to 7). In other words, the reflector 73 of the reflector unit 7 reflects the illumination light LL and the observation light OL so that the angle of each of the illumination unit 3, the camera unit 4L, and the camera unit 4R relative to the eyeball 200 can be reduced. This configuration solves the problem that imaging is difficult due to the feature of the face of the subject as described above. It is possible to clearly image the crystalline lens of the eyeball 200 including the equator, for any subject. The same applies to a region of the eyeball 200 other than the crystalline lens, for example, the Zinn's zonule.

Note that only one of the camera unit 4L and the camera unit 4R may be used. Specifically, in a case where the three-dimensional shape of the region of the eyeball 200 as the target to be imaged is grasped, both of the camera unit 4L and the camera unit 4R are used. Otherwise, it is only necessary to use only one of the camera unit 4L and the camera unit 4R.

FIG. 11 illustrates diagrams of exemplary shapes of the reflector unit. Some examples of the shapes of the extending portion 722 and the reflector 73 when viewing the extending portion 722 from the base portion 721 of the main body 72 are illustrated.

The extending portion 722 of the reflector unit 7 illustrated in (A) of FIG. 11 has a shape in which a conical distal end portion is removed. The reflector 73 is provided over the entire side surface of the extending portion 722 so as to fit to the side surface of the extending portion 722. This configuration makes it possible to capture an image of the eyeball 200 while rotationally moving the illumination unit 3, the camera unit 4L, and the camera unit 4R with the reflector unit 7 fixed. Although there is a possibility that the image is distorted, when the image is distorted, the processing unit 22 of the control processing device 20 preferably performs correction processing.

The extending portion 722 of the reflector unit 7 illustrated in (B) of FIG. 11 has a shape in which a polygonal pyramidal distal end portion is removed. The reflector 73 is provided over the entire side surface of the extending portion 722 so as to fit to the side surface of the extending portion 722. This configuration also makes it possible to capture an image of the eyeball 200 while rotationally moving the illumination unit 3, the camera unit 4L, and the camera unit 4R with the reflector unit 7 fixed. Although there is a possibility that discontinuity of the images may occur at a joint portion between the surfaces of the reflector 73, the processing unit 22 of the eyeball 200 preferably perform processing of accommodating the discontinuity.

The extending portion 722 of the reflector unit 7 illustrated in (C) of FIG. 11 has a shape in which a conical distal end portion is truncated. The reflector 73 has a flat shape and is provided on a part of the side surface of the extending portion 722. In this example, one reflector 73 is provided, but two or more reflectors 73 may be provided. Rotational movement of the reflector unit 7 together with the illumination unit 3, the camera unit 4L, and the camera unit 4R makes it possible to image the eyeball 200. A clear image of wide range without distortion is obtained at each angle.

The reflector unit 7 may be used in contact with the eyeball 200, or may be used away from the eyeball 200 (non-contact state). When the reflector unit 7 is used in contact with the eyeball 200, the reflector unit 7 is not affected by corneal surface aberration and further is not affected by a critical angle at an interface between the cornea and air, thus enabling observation through a larger angle. For the invasiveness to the eyeball 200, it is possible to cope with the invasiveness by anesthesia or the like at the time of examination. When the reflector unit 7 is used in the non-contact state, there is no problem of the invasiveness.

### 1.8. Fixation Target

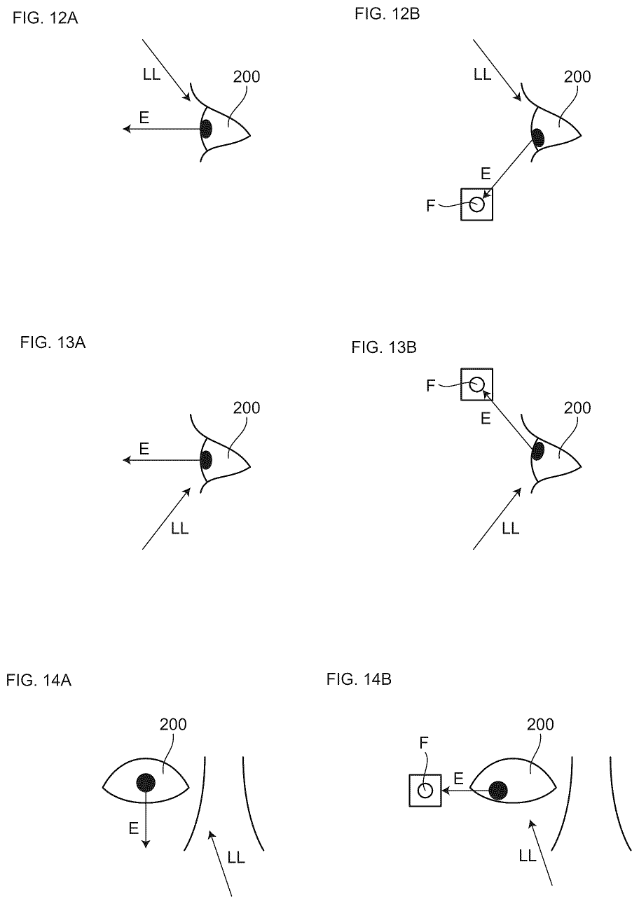

Depending on the shape or the like of the face of the subject, it may be still difficult to image the lens equator or Zinn's zonule of the eyeball 200. This problem can be solved by changing the orientation of the eyeball 200, that is, a line of sight of the subject. In an embodiment, the eyeball 200 may be imaged, with the fixation target for guiding the orientation of the eyeball 200 presented to the subject. The fixation target will be described with reference to FIGS. 12 to 14.

FIGS. 12 to 14 are each a diagram illustrating an example of presentation of the fixation target. The line of sight of the subject that corresponds to the orientation of the eyeball 200 is illustrated as a line of sight E and indicated by an arrow. The fixation target is illustrated as fixation target F.

FIG. 12 illustrates an example of the fixation

target F that guides the line of sight E downward. As illustrated in (A) of FIG. 12, when the line of sight E is directed straight forward, the illumination light LL is blocked by an upper eyelid, the forehead, and the like, and the eyeball 200 cannot be imaged. As illustrated in (B) of FIG. 12, the fixation target F is displayed below the eyeball 200. The line of sight E moves downward, and accordingly, the illumination light LL is emitted from a different position or angle. The illumination light LL reaches the eyeball 200 without being blocked, and the eyeball 200 is imaged.

FIG. 13 illustrates an example of the fixation target F that guides the line of sight E upward. As illustrated in (A) of FIG. 13, when the line of sight E is directed straight forward, the illumination light LL is blocked by a lower eyelid or the like, and the eyeball 200 cannot be imaged. As illustrated in part (B) of FIG. 13, the fixation target F is displayed above the eyeball 200. The line of sight E moves upward, and accordingly, the illumination light LL is emitted from a different position or angle. The illumination light LL reaches the eyeball 200 without being blocked, and the eyeball 200 is imaged.

FIG. 14 illustrates an example of the fixation target F that guides the line of sight E laterally. As illustrated in (A) of FIG. 14, when the line of sight E is directed straight forward, the illumination light LL is blocked by a nose or the like, and the eyeball 200 cannot be imaged. As illustrated in (B) of FIG. 14, the fixation target F is displayed on a lateral side of the eyeball 200. The line of sight E moves laterally, and accordingly, the illumination light LL is emitted from a different position or angle. The illumination light LL reaches the eyeball 200 without being blocked, and the eyeball 200 is imaged.

Note that a device (fixation target device) for presenting the fixation target F to the subject may also be a component element of the imaging device 10. In a state where the fixation target F is presented to the subject, the illumination unit 3 illuminates the eyeball 200, and at least one of the camera unit 4L and the camera unit 4R images the eyeball 200. This configuration makes it possible to avoid difficulty in imaging of the region of the eyeball 200 as the target to be imaged due to the shape of the face of the subject or the like. All the examinations can be performed comprehensively, regardless of the shape of the face of the subject or the like.

When the fixation target F is used as described above, the region of the eyeball 200 is imaged in a state where the eyeball 200 faces in a direction different from a normal direction. The processing unit 22 of the control processing device 20 integrates an image obtained by imaging in such a state where the eyeball 200 faces in a direction different from the normal direction and another image captured in a normal state. For example, on the basis of features obtained from the images, the regions shown in the images are aligned. Examples of the features include texture features, shape features, and the like of iris, blood vessel, tissue, and the like.

### 1.9. Image Processing

Furthermore, a description will be made of image processing (signal processing) by the processing unit 22 of the control processing device 20. The processing unit 22 of the control processing device 20 calculates the shape of each of the regions of the eyeball 200, the opacity distribution, or the like, on the basis of each image from the imaging device 10. For example, the shapes of an anterior surface of cornea, a posterior surface of cornea, the crystalline lens (including the equator), and the like are calculated, and the opacity distribution in the crystalline lens is calculated.

Many of the regions of the eyeball 200 have a refractive index different from that of air. When the illumination light LL or the observation light OL (hereinafter, also simply referred to as “a ray of light”) passes through such a region, the ray of light is refracted. A captured image of each region of the eyeball 200 can include the influence of refraction of the ray of light. Performing ray tracing in consideration of the refraction of the ray of light makes it possible to calculate the shape of each region more accurately.

The ray tracing in consideration of the refraction is performed, for example, on the basis of (a) the shape of a region on which the ray of light is incident, (b) a position and a direction of incidence of the ray of light, and (c) the refractive indices of the region before and after the incidence of the ray of light. If (a) is grasped, (b) can also be grasped, and a representative value or an actual measurement value can be used for (c). Therefore, it is particularly important to grasp (a).

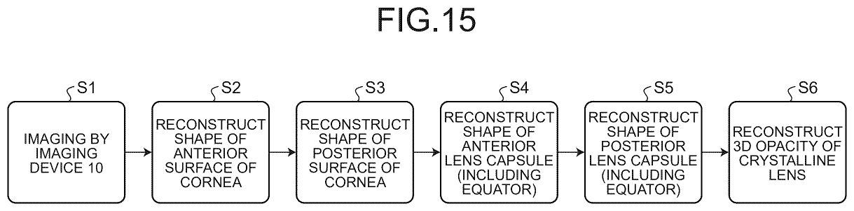

Before calculating the shape of a region of the eyeball 200, the processing unit 22 of the control processing device 20 calculates the shape of another region positioned in front (near the imaging device 10) of the region first. Then, as described in (a) to (c), the processing unit 22 performs ray tracing in consideration of the shape of the another region on the front side, the position and direction of incidence of the ray of light, and the refraction of the ray of light, and thereafter, calculates the shape of the region positioned in back of the another region. This configuration makes it possible to perform calculation with higher accuracy than that of calculation without consideration of the refraction of the ray of light. A specific example will be described with reference to FIGS. 15 to 20.

FIG. 15 is a flowchart illustrating an example of image processing. In this example, the three-dimensional shape of the crystalline lens of the eyeball 200 and the three-dimensional opacity distribution in the crystalline lens are finally calculated. Unless otherwise specified, each process is performed by the processing unit 22 of the control processing device 20.

In Step S1, the eyeball 200 is imaged by the imaging device 10. Here, it is assumed that imaging related to the corneal examination and the crystalline lens examination is performed. Details have been described above, and repetitive description will be omitted.

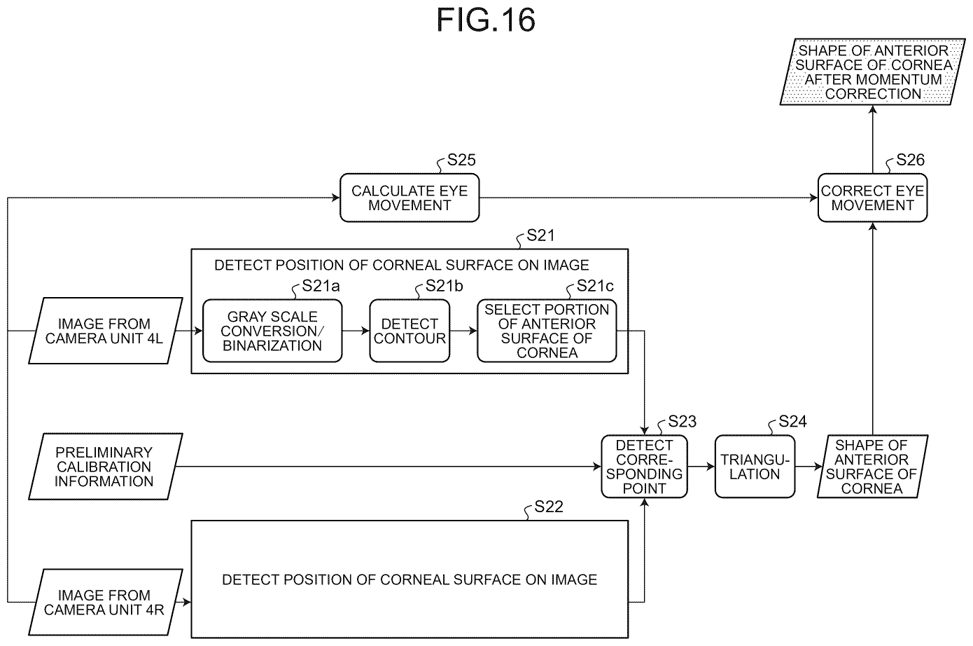

In Steps S2 to S5, the shapes of the anterior surface of cornea, posterior surface of cornea, anterior lens capsule including the equator, and posterior lens capsule including the equator are reconstructed (calculated) in this order. These regions are positioned in the order of passage of the ray of light reaching the posterior lens capsule. Furthermore, in Step S6, the three-dimensional opacity of the crystalline lens is reconstructed (opacity distribution is calculated). Details of Steps S2 to S6 will be described with reference to FIGS. 16 to 20.

FIG. 16 is an exemplary flowchart illustrating Step S2 of FIG. 15 in detail. In Step S21, the position of the corneal surface on the image is detected on the basis of the image of the eyeball 200 captured by the camera unit 4L. As specific processing, gray scale conversion/binarization in Step S21a, contour extraction in Step S21b, and selection of the portion of the anterior surface of cornea in Step S21c are exemplified. A technique of detecting the position of an object on an image by such processing is publicly known, and thus, a detailed description thereof will be omitted.

In Step S22, the position of the corneal surface on the image is detected on the basis of the image of the eyeball 200 captured by the camera unit 4R. The specific processing is similar to Step S21 described above. Note that, hereinafter, the image of the eyeball 200 captured by the camera unit 4L is also simply referred to as the image from the camera unit 4L. The image of the eyeball 200 captured by the camera unit 4R is also simply referred to as the image from the camera unit 4R.

In Step S23, a corresponding point is detected on the basis of a result of the detection of the position of the corneal surface in previous Steps S21 and S22 and preliminary calibration information. Examples of the preliminary calibration information include the internal parameters and external parameters of the illumination unit 3, the camera unit 4L, and the camera unit 4R described above. On the basis of the preliminary calibration information, each position (each point) in the image from the camera unit 4L and corresponding each position (each point) in the image from the camera unit 4R are detected.

In Step S24, triangulation is performed. A distance between the corresponding points detected in Step S24 described above is measured. The triangulation itself is publicly known, and detailed description thereof will be omitted. The shape of the anterior surface of cornea is calculated on the basis of a result of the triangulation.

In Step S25, eye movement is calculated on the basis of the image from the camera unit 4L and the image from the camera unit 4R. A change in orientation of the eyeball 200 and the like during imaging are calculated.

In Step S26, eye movement correction is performed. The shape of the anterior surface of cornea calculated on the basis of the result of the triangulation in Step S24 described above is corrected on the basis of the eye movement calculated in Step S25 described above. An influence such as a change in orientation of the eyeball 200 during imaging is removed. The shape of the anterior surface of cornea after momentum correction is calculated.

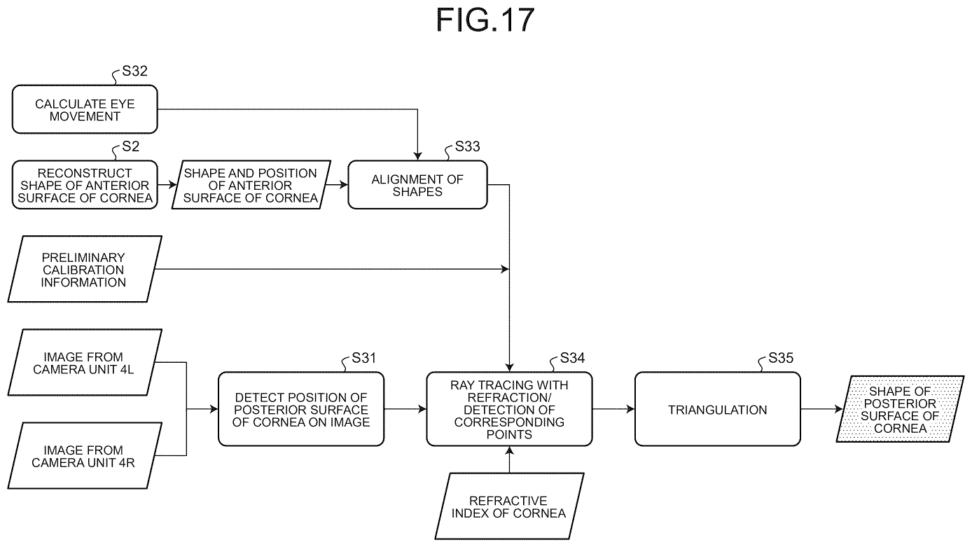

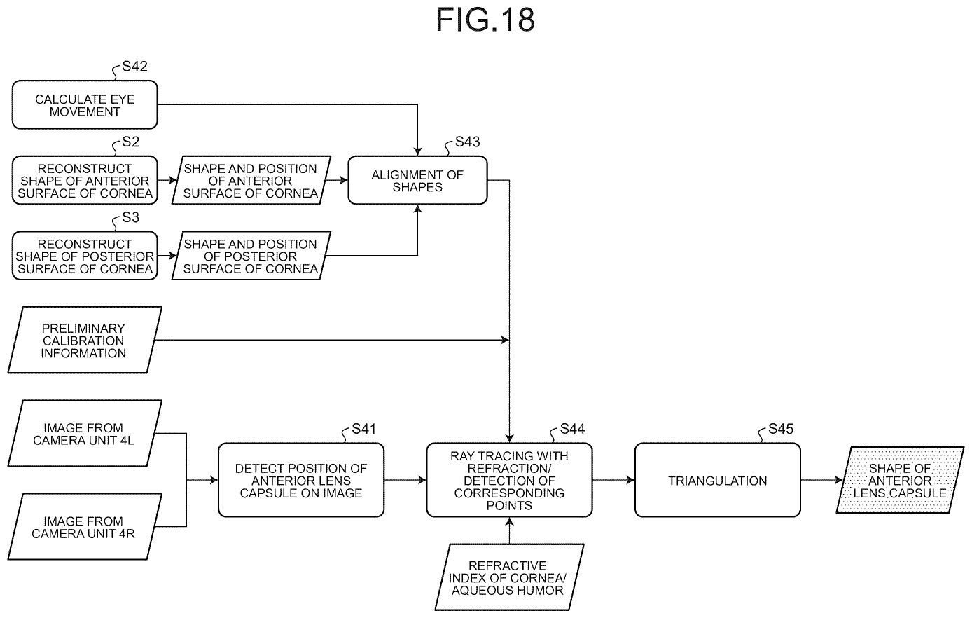

FIG. 17 is an exemplary flowchart illustrating Step S3 of FIG. 15 in detail. The process of FIG. 17 is different from the process of FIG. 16 described above particularly in that refraction of the ray of light passing through a region that is positioned in front and whose shape has been calculated is taken into consideration. The same applies to FIGS. 18 to 20 described later. Repetitive descriptions will be omitted as appropriate.

In Step S31, the position of the posterior surface of cornea on the image is detected on the basis of the image from the camera unit 4L and the image from the camera unit 4R. In Step S32, eye movement is calculated. In Step S33, the shapes of the respective regions are aligned on the basis of the shape and position of the anterior surface of cornea calculated in Step S2 described above and the eye movement calculated in Step S32 described above.

In Step S34, ray tracing in consideration of refraction (ray tracing with refraction) is performed on the basis of the position of the posterior surface of cornea detected in Step S31 described above, the preliminary calibration information, the shapes of the respective regions aligned in Step S33 described above, and the refractive index of the cornea, and the corresponding points are detected. In Step S35, triangulation is performed. A distance between the respective corresponding points detected in Step S34 described above is measured, and the shape of the posterior surface of cornea is calculated.

FIG. 18 is an exemplary flowchart illustrating Step S4 of FIG. 15 in detail. In Step S41, the position of the anterior lens capsule on the image is detected on the basis of the image from the camera unit 4L and the image from the camera unit 4R. In Step S42, eye movement is calculated. In Step S43, the shapes of the respective regions are aligned on the basis of the shape and position of the anterior surface of cornea calculated in Step S2 described above, the shape and position of the posterior surface of cornea calculated in Step S3 described above, and the eye movement calculated in Step S42 described above.

In Step S44, ray tracing in consideration of refraction is performed on the basis of the position of the anterior lens capsule detected in Step S41 described above, the preliminary calibration information, the shapes of the respective regions aligned in Step S43 described above, and the refractive indices of the cornea and aqueous humor, and the corresponding points are detected. In Step S45, triangulation is performed. A distance between the respective corresponding points detected in Step S44 described above is measured, and the shape of the anterior lens capsule is calculated.

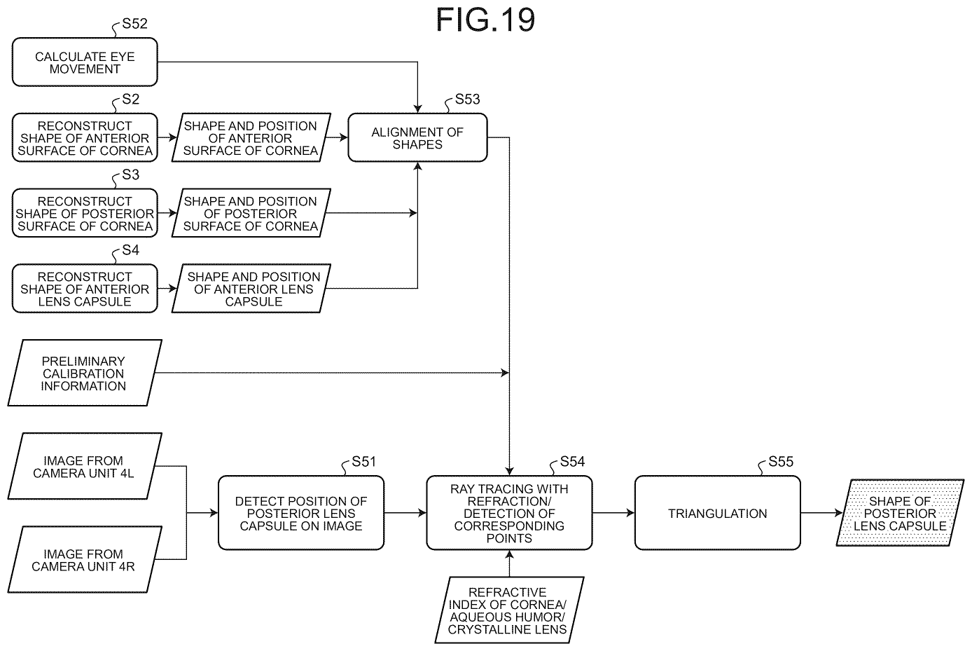

FIG. 19 is an exemplary flowchart illustrating Step S5 of FIG. 15 in detail. In Step S51, the position of the posterior lens capsule on the image is detected on the basis of the image from the camera unit 4L and the image from the camera unit 4R. In Step S52, eye movement is calculated. In Step S53, the shapes of the respective regions are aligned on the basis of the shape and position of the anterior surface of cornea calculated in Step S2 described above, the shape and the position of the posterior surface of cornea calculated in Step S3 described above, the shape and position of the anterior lens capsule calculated in Step S4 described above, and the eye movement calculated in Step S52 described above.

In Step S54, ray tracing in consideration of refraction is performed on the basis of the position of the posterior lens capsule detected in Step S51 described above, the preliminary calibration information, the shape of the respective regions aligned in Step S53 described above, and the refractive indices of the cornea, aqueous humor, and crystalline lens, and the corresponding points are detected. In Step S55, triangulation is performed. A distance between the respective corresponding points detected in Step S44 described above is measured, and the shape of the posterior lens capsule is calculated.

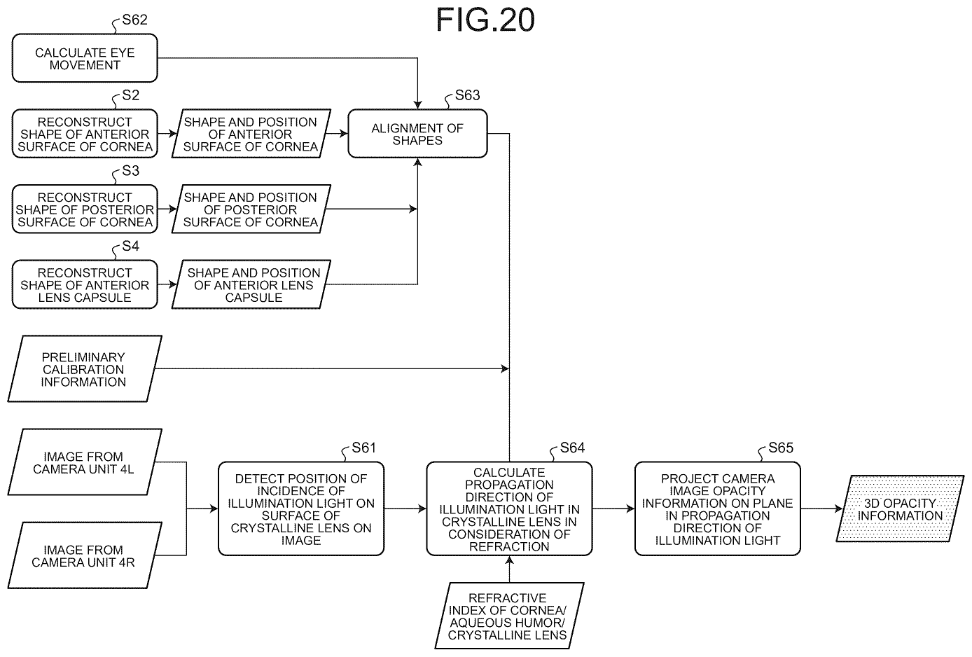

FIG. 20 is an exemplary flowchart illustrating Step S6 of FIG. 15 in detail. In Step S61, the position of incidence of the illumination light LL on the surface of the crystalline lens on the image is detected on the basis of the image from the camera unit 4L and the image from the camera unit 4R. In Step S62, eye movement is calculated. In Step S63, the shapes of the respective regions are aligned on the basis of the shape and position of the anterior surface of cornea calculated in Step S2 described above, the shape and position of the posterior surface of cornea calculated in Step S3 described above, the shape and position of the anterior lens capsule calculated in Step S4 described above, and the eye movement calculated in Step S62 described above.

In Step S64, a propagation direction of the illumination light LL in the crystalline lens in consideration of refraction is calculated, on the basis of the position of incidence of the illumination light LL on the surface of the crystalline lens detected in Step S61 described above, the preliminary calibration information, the shape of the respective regions aligned in Step S63 described above, and the refractive indices of the cornea, aqueous humor, and crystalline lens. In Step S65, opacity appearing in a camera image (camera image opacity information) is projected on a plane in the propagation direction of the illumination light LL, and the three-dimensional opacity distribution in the crystalline lens is calculated. Three-dimensional opacity information is obtained.

For example, the three-dimensional shape and opacity distribution of a desired region of the eyeball 200 are allowed to be calculated as described above. The ray tracing in consideration of refraction of the ray of light passing through the region positioned in front makes it possible to accurately calculate the shape and opacity distribution of the region of the eyeball 200, as compared with calculation without consideration of refraction of the ray of light.

### 2. Modifications

The disclosed technology is not limited to the above embodiments. For example, in the above embodiments, the example of the imaging device 10 including two camera units of the camera unit 4L and the camera unit 4R has been described. However, the number of camera units may be one.

When only one of the camera unit 4L and the camera unit 4R is used, the roles of the camera unit 4L and the camera unit 4R described in the above embodiments may be exchanged.

In the above embodiments, the anterior segment, more specifically, the gonioscope region, cornea, crystalline lens, Zinn's zonule, and corneal endothelial cells have been described as examples of the region of the eyeball 200 as the target to be examined by the examination system 100. As a matter of course, the other regions of the eyeball 200 may also be included in the target to be examined by the examination system 100.

Instead of movement of the measurement unit 2 to change the angle relative to the eyeball 200 as indicated by the arrow AR2A, the illumination unit 3, the camera unit 4L, and the camera unit 4R may be moved together as indicated by the arrow AR3, the arrow AR4L, and the arrow AR4R. The movement of the measurement unit 2 to change the angle relative to the eyeball 200 may be replaced with the movement of the illumination unit 3, the camera unit 4L, and the camera unit 4R together in order to change the angles relative to the eyeball 200, as long as there is no contradiction.

### 3. Exemplary Hardware Configuration

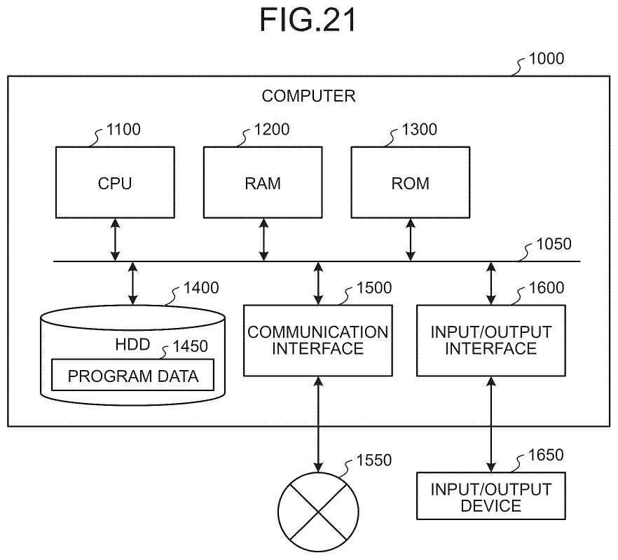

FIG. 21 is a diagram illustrating an exemplary hardware configuration of the control processing device 20. The control processing device 20 described above is implemented by including, for example, a computer 1000 as illustrated in FIG. 21.

The computer 1000 includes a CPU 1100, a RAM 1200, a read only memory (ROM) 1300, a hard disk drive (HDD) 1400, a communication interface 1500, and an input/output interface 1600. The respective units of the computer 1000 are connected by a bus 1050.

The CPU 1100 is operated on the basis of programs stored in the ROM 1300 or the HDD 1400 to control the respective units. For example, the CPU 1100 deploys a program (program 231 or the like in FIG. 1) stored in the ROM 1300 or the HDD 1400 to the RAM 1200, and performs processing corresponding to each of various programs.

The ROM 1300 stores a boot program, such as a basic input output system (BIOS), executed by the CPU 1100 when the computer 1000 is booted, a program depending on the hardware of the computer 1000, and the like.

The HDD 1400 is a computer-readable recording medium that non-transitorily records a program executed by the CPU 1100, data used by the program, and the like. For example, the HDD 1400 is a recording medium corresponding to the storage unit 23 of the control processing device 20 (FIG. 1) and recording the program 231.

The communication interface 1500 is an interface for connecting the computer 1000 with an external network 1550 (e.g., the Internet). For example, the CPU 1100 receives data from another device or transmits data generated by the CPU 1100 to the another device, via the communication interface 1500.

The input/output interface 1600 is an interface for connecting an input/output device 1650 and the computer 1000. For example, the CPU 1100 receives data from an input device such as a keyboard or mouse via the input/output interface 1600. In addition, the CPU 1100 transmits data to an output device such as a display, speaker, or printer via the input/output interface 1600. Furthermore, the input/output interface 1600 may function as a media interface that reads a program or the like recorded on a predetermined recording medium. The medium includes, for example, an optical recording medium such as a digital versatile disc (DVD) or phase change rewritable disk (PD), a magneto-optical recording medium such as a magneto-optical disk (MO), a tape medium, a magnetic recording medium, a semiconductor memory, or the like.

For example, when the computer 1000 functions as the imaging device 10 according to an embodiment, the CPU 1100 of the computer 1000 executes a program loaded on the RAM 1200 to implement a function of the control processing device 20. Furthermore, the HDD 1400 stores the program 231, or data in the storage unit 23. Note that the CPU 1100 executes program data 1450 read from the HDD 1400, but in another example, the CPU 1100 may acquire these programs from another device via the external network 1550.

### 4. Exemplary Effects

For example, the technologies described above are specified as follows. One of the disclosed technologies is the examination system 100. As described with reference to FIGS. 1 and 2 and the like, the examination system 100 includes the measurement unit 2, the illumination unit 3, the camera unit 4L, and the camera unit 4R. The measurement unit 2 is movable so as to change the angle relative to the eyeball 200 of the subject (arrow AR2A), and rotatably moved around the eye axis of the eyeball as the rotation axis (arrow AR2B). The illumination unit 3 is mounted on the measurement unit 2 to irradiate the eyeball 200. The camera unit 4L is the first camera unit that is mounted on the measurement unit 2 and images the eyeball 200. The camera unit 4R is the second camera unit that is mounted on the measurement unit 2 and images the eyeball 200. The camera unit 4L and the camera unit 4R are independently movable on the measurement unit 2 (arrow AR4L and arrow AR4R) to change the angles relative to the eyeball 200.

According to the above examination system 100, the measurement unit 2, the illumination unit 3, the camera unit 4L, and the camera unit 4R are independently movable. Combining the respective movements makes it possible to image the eyeball 200 in various modes suitable for various examinations.

As described with reference to FIGS. 1 and 2 and the like, in a state where the camera unit 4L and the camera unit 4R are arranged on the opposite sides across the illumination unit 3, the measurement unit 2 may move to change the angle relative to the eyeball 200 (arrow AR2A), and the camera unit 4L and the camera unit 4R may image the anterior segment of the eyeball 200. Thus, the anterior segment of the eyeball 200 is allowed to be examined.

As described with reference to FIGS. 2 and 3 and the like, the examination system 100 may include the gonioscope unit 5 that is movable (arrow AR5) between the position (insertion position) located between the eyeball 200, and, the illumination unit 3 and at least one (e.g., the camera unit 4L) of the camera unit 4L and the camera unit 4R, and the other position (retraction position). In this configuration, the measurement unit 2 may rotationally move around the eye axis EA of the eyeball 200 as the rotation axis (arrow AR2B) and at least one of the camera units (e.g., the camera unit 4L) may image the angle region of the eyeball 200, in a state where the gonioscope unit 5 is arranged at the position between the eyeball 200, and, the illumination unit 3 and the at least one of the camera units (e.g., the camera unit 4L). In this configuration, the examination system 100 may include the reflective optical system unit 6 that is movable between the position between the gonioscope unit 5, and, the illumination unit 3 and at least one of the camera units (e.g., the camera unit 4L), and the other position. In this configuration, the measurement unit may rotationally move around the eye axis EA of the eyeball 200 as the rotation axis (arrow AR2B), and at least one of the camera units (e.g., the camera unit 4L) may image the angle region of the eyeball 200, in a state where the reflective optical system unit 6 is arranged at the position between the gonioscope unit 5, and the illumination unit 3 and the at least one of the camera units (e.g., camera unit 4L), This configuration makes it possible to examine the angle region of the eyeball 200.

As described with reference to FIG. 4 and the like, in a state where the illumination unit 3 is arranged in front of the eyeball 200 and further the camera unit 4L and the camera unit 4R are arranged on the opposite sides across the illumination unit 3 to have angles of the same magnitude relative to the eyeball 200, the measurement unit 2 may rotationally move around the eye axis EA of the eyeball 200 as the rotation axis (arrow AR2B), and the camera unit 4L and the camera unit 4R may image the cornea of the eyeball 200. This configuration makes it possible to examine the cornea of the eyeball 200.

As described with reference to FIGS. 5 to 7 and the like, the crystalline lens of the eyeball 200 can also be examined. For example, as described with reference to FIG. 5, in a state where the illumination unit 3 is arranged at the position where the illumination unit 3 has an angle relative to the eyeball 200, the measurement unit 2 may rotationally move around the eye axis EA of the eyeball 200 as the rotation axis (arrow AR2B), and the camera unit 4L and the camera unit 4R may image the crystalline lens of the eyeball 200. This configuration makes it possible to grasp the shape of the crystalline lens of the eyeball 200 including the equator. As described with reference to FIGS. 2 and 6 and the like, similarly to the camera unit 4L and the camera unit 4R, the illumination unit 3 is also independently movable on the measurement unit 2 to change the angle relative to the eyeball 200, and in a state where the illumination unit 3 is arranged at the position where the illumination unit 3 has an angle relative to the eyeball 200, the illumination unit 3, the camera unit 4L, and the camera unit 4R may move together to change the angles relative to the eyeball 200 while maintaining the positional relationship therebetween, and the camera unit 4L and the camera unit 4R may image the crystalline lens of the eyeball 200. In this configuration, as described with reference to FIG. 7, the measurement unit 3 may further rotationally move around the eye axis EA of the eyeball 200 as the rotation axis, and the camera unit 4L and the camera unit 4R may image the crystalline lens of the eyeball 200. This configuration makes it possible to grasp the opacity distribution of the crystalline lens of the eyeball 200.

As described with reference to FIG. 8 and the like, in a state where the illumination unit 3 and at least one of the camera unit 4L and the camera unit 4R (e.g., the camera unit 4L) are arranged at the positions where the illumination unit 3 and the at least one of the camera units have angles relative to the eyeball 200, the measurement unit 2 may rotationally move around the eye axis EA of the eyeball 200 as the rotation axis (arrow AR2B), and at least one of the camera units (e.g., the camera unit 4L) may image the Zinn's zonule of the eyeball 200. This configuration makes it possible to examine the Zinn's zonule of the eyeball 200.

As described with reference to FIGS. 1 and 9 and the like, the camera unit 4L includes the objective zoom optical system 41, the camera unit 4R includes the objective zoom optical system 41, the illumination unit 3 is arranged at the position where the illumination unit 3 has an angle relative to the line normal to the corneal endothelium as the target to be imaged in the eyeball 200, the camera unit 4L is arranged in front of the corneal endothelium as the target to be imaged, the camera unit 4R is arranged on a side opposite from the illumination unit 3 across the line normal to the corneal endothelium as the target to be imaged, axially symmetrically. In such a state, the camera unit 4L may image the entire cornea of the eyeball 200, the measurement unit 2 may move to change the angle relative to the eyeball 200 and the measurement unit 2 may rotationally move around the eye axis EA of the eyeball 200 as the rotation axis (arrows AR2A and AR2B), and the camera unit 4R may image the corneal endothelial cells of the eyeball 200. This configuration makes it possible to examine the corneal endothelial cells of the eyeball 200.

As described with reference to FIGS. 10 and 11 and the like, the examination system 100 may include the reflector unit 7 that is movable between the position between the eyeball 200, and, the illumination unit 3 and at least one of the camera unit 4L and the camera unit 4R (e.g., the camera unit 4L and the camera unit 4R), and the other position. In this configuration, in a state where the reflector unit 7 is arranged at the position between the eyeball 200, and the illumination unit 3 and at least one of the camera units (e.g., the camera unit 4L and the camera unit 4R), each of the illumination unit 3 and the at least one of camera units (e.g., the camera unit 4L and the camera unit 4R) may be arranged at the position where the angle relative to the eyeball 200 is smaller than that of a configuration without the reflector unit 7. This configuration solves the problem that imaging is difficult due to the feature of the face of the subject.

As described with reference to FIG. 12 and the like, in a state where the fixation target F for guiding the orientation of the eyeball 200 (line of sight E) is presented to the subject, the illumination unit 3 may illuminate the eyeball 200, and at least one of the camera unit 4R and the camera unit 4L may image the eyeball 200. This configuration makes it possible to perform all examinations comprehensively, regardless of the shape or the like of the face of the subject.

As described with reference to FIGS. 1 and 2 and the like, the examination system 100 may include the base unit 1 that is movable forward and backward (arrow AR1A), leftward and rightward (arrow AR1B), and vertically (arrow AR1C) relative to the eyeball 200, and the measurement unit 2 may be mounted on the base unit 1. This configuration facilitates alignment of the respective units on the base unit 1 with positions suitable for examinations of the eyeball 200.

As described with reference to FIG. 1 and the like, the illumination unit 3 may irradiate the eyeball 200 with the illumination light LL, and the illumination light LL may include the slit lamp light. This configuration makes it possible to perform examination using the slit lamp light.

As described with reference to FIGS. 1, 15 to 20, and the like, the examination system 100 may include the processing unit 22 that calculates the shape of a region of the eyeball 200 on the basis of an image of the eyeball 200 captured by at least one of the camera unit 4L and the camera unit 4R, and in this configuration, the processing unit 22 may calculate the shape of another region located in front of the region of the eyeball 200 and then calculate the shape of the region located in back of the another region on the basis of ray tracing in consideration of the refraction of the ray of light passing through the shape of the another region. For example, the processing unit 22 may sequentially calculate the shape of the anterior surface of cornea of the eyeball 200, the shape of the posterior surface of cornea of the eyeball 200, the shape of the anterior lens capsule of the eyeball 200, and the shape of the posterior lens capsule of the eyeball 200 to calculate the shape of the crystalline lens of the eyeball 200. The processing unit 22 may calculate the opacity distribution in the crystalline lens of the eyeball 200. This configuration makes it possible to calculate the shape and opacity distribution of the region of the eyeball 200 with higher accuracy than that of calculation without consideration of the refraction of the ray of light.

Note that the effects described in the present disclosure are merely examples and the effects are not limited to those disclosed. Other effects may be provided.

The embodiments of the present disclosure have been described above, but the technical scope of the present disclosure is not strictly limited to the embodiments described above, and various modifications and alterations can be made without departing from the spirit and scope of the present disclosure. Moreover, the component elements of different embodiments and modifications may be suitably combined with each other.

Note that the present technology can also have the following configurations.

(1) An examination system comprising:
a measurement unit that is movable to change an angle relative to an eyeball of a subject and rotatably moved around an eye axis of the eyeball as a rotation axis;
an illumination unit that is mounted on the measurement unit to irradiate the eyeball;
a first camera unit that is mounted on the measurement unit to image the eyeball; and
a second camera unit that is mounted on the measurement unit to image the eyeball, wherein
the first camera unit and the second camera unit are independently movable on the measurement unit to change angles relative to the eyeball.

(2) The examination system according to (1), wherein
in a state where the first camera unit and the second camera unit are arranged on opposite sides across the illumination unit,
the measurement unit is moved to change the angle relative to the eyeball, and
the first camera unit and the second camera unit image an anterior segment of the eyeball.

(3) The examination system according to (1) or (2), further comprising
a gonioscope unit that is movable between a position between the eyeball, and the illumination unit and at least one of the first camera unit and the second camera unit, and another position, wherein
in a state where the gonioscope unit is arranged at a position between the eyeball, and the illumination unit and the at least one of the first and second camera units,
the measurement unit rotationally moves around the eye axis of the eyeball as the rotation axis, and
the at least one of the first and second camera units images an angle region of the eyeball.

(4) The examination system according to (3), further comprising
a reflective optical system unit that is movable between a position between the gonioscope unit, and the illumination unit and the at least one of the first and second camera units, and another position, wherein
in a state where the reflective optical system unit is arranged at a position between the gonioscope unit, and the illumination unit and the at least one of the first and second camera units,
the measurement unit rotationally moves around the eye axis of the eyeball as the rotation axis, and
the at least one of the first and second camera units images the angle region of the eyeball.

(5) The examination system according to any one of (1) to (4), wherein
in a state where the illumination unit is arranged in front of the eyeball, and
where the first camera unit and the second camera unit are arranged on opposite sides across the illumination unit to have angles of the same magnitude relative to the eyeball,
the measurement unit rotationally moves around the eye axis of the eyeball as the rotation axis, and
the first camera unit and the second camera unit image a cornea of the eyeball.

(6) The examination system according to any one of (1) to (5), wherein
in a state where the illumination unit is arranged at a position where the illumination unit has an angle relative to the eyeball,
the first camera unit and the second camera unit move together to change angles of the camera units relative to the eyeball while maintaining an inward angle between the camera units,
the first camera unit and the second camera unit image a crystalline lens of the eyeball,
the measurement unit rotationally moves around an eye axis of the eyeball as a rotation axis, and
the first camera unit and the second camera unit image the crystalline lens of the eyeball.

(7) The examination system according to any one of (1) to (6), wherein
the illumination unit is also independently movable on the measurement unit to change an angle relative to the eyeball, as in the first camera unit and the second camera unit, and
in a state where the illumination unit is arranged at a position where the illumination unit has an angle relative to the eyeball,
the illumination unit, the first camera unit, and the second camera unit move together to change angles relative to the eyeball while maintaining a positional relationship between the illumination unit and the first and second camera units, and
the first camera unit and the second camera unit image a crystalline lens of the eyeball.

(8) The examination system according to (7), wherein
the measurement unit further rotationally moves around the eye axis of the eyeball as the rotation axis, and
the first camera unit and the second camera unit image the crystalline lens of the eyeball.

(9) The examination system according to any one of (1) to (8), wherein
in a state where the illumination unit, and at least one of the first camera unit and the second camera unit are arranged at positions where the illumination unit and the at least one of the first and second camera units have angles relative to the eyeball,
the measurement unit rotationally moves around the eye axis of the eyeball as the rotation axis, and
the at least one of the first and second camera units images Zinn's zonule of the eyeball.

(10) The examination system according to any one of (1) to (9), wherein
the first camera unit includes an objective zoom optical system,
the second camera unit includes an objective zoom optical system, and
in a state where the illumination unit is arranged at a position where the illumination unit has an angle relative to a line normal to a corneal endothelium as a target to be imaged in the eyeball, where the first camera unit is arranged in front of the corneal endothelium as the target to be imaged, and where the second camera unit is arranged on a side opposite from the illumination unit across the line normal to the corneal endothelium as the target to be imaged, axially symmetrically,
the first camera unit images an entire cornea of the eyeball,
the measurement unit moves to change an angle relative to the eyeball, and the measurement unit rotationally moves around the eye axis of the eyeball as the rotation axis, and
the second camera unit images corneal endothelial cells of the eyeball.

(11) The examination system according to any one of (1) to (10), further comprising
a reflector unit that is movable between a position between the eyeball, and, the illumination unit and at least one of the first camera unit and the second camera unit, and another position, wherein
in a state where the reflector unit is arranged at a position between the eyeball, and the illumination unit and the at least one of the first and second camera units,
each of the illumination unit and the at least one of the first and second camera units is arranged at a position where an angle relative to the eyeball is smaller than an angle of a configuration without the reflector unit.

(12) The examination system according to any one of (1) to (11), wherein
in a state where a fixation target for guiding an orientation of the eyeball is presented to the subject,
the illumination unit illuminates the eyeball, and
at least one of the first camera unit and the second camera unit images the eyeball.

(13) The examination system according to any one of (1) to (12), further comprising
a base unit that is movable forward and backward, leftward and rightward, and vertically, relative to the eyeball, wherein
the measurement unit is mounted on the base unit.

(14) The examination system according to any one of (1) to (13), wherein
the illumination unit irradiates the eyeball with illumination light, and
the illumination light includes slit lamp light.

(15) The examination system according to any one of (1) to (14), further comprising
a processing unit that calculates shapes of regions of the eyeball based on an image of the eyeball imaged by at least one of the first camera unit and the second camera unit, wherein
the processing unit calculates a shape of a region of the regions of the eyeball, positioned in front, and based on ray tracing in consideration of refraction of a ray of light passing through the calculated shape, calculates a shape of a portion positioned in back of the region in front.

(16) The examination system according to (15), wherein
the processing unit sequentially calculates a shape of an anterior surface of cornea of the eyeball, a shape of a posterior surface of cornea of the eyeball, a shape of an anterior lens capsule of the eyeball, and a shape of a posterior lens capsule of the eyeball to calculate a shape of a crystalline lens of the eyeball.

(17) The examination system according to (16), wherein
the processing unit calculates an opacity distribution in the crystalline lens of the eyeball.

### REFERENCE SIGNS LIST

100 EXAMINATION SYSTEM
10 IMAGING DEVICE
1 BASE UNIT
2 MEASUREMENT UNIT
3 ILLUMINATION UNIT
31 LIGHT SOURCE
32 SLIT
33A OPTICAL SYSTEM
33B OPTICAL SYSTEM
4L CAMERA UNIT (FIRST CAMERA UNIT)
4R CAMERA UNIT (SECOND CAMERA UNIT)
41 OBJECTIVE ZOOM OPTICAL SYSTEM
42 IMAGE SENSOR
5 GONIOSCOPE UNIT
6 REFLECTIVE OPTICAL SYSTEM UNIT
7 REFLECTOR UNIT
71 FRAME BODY
72 MAIN BODY
73 REFLECTOR
20 CONTROL PROCESSING DEVICE
21 CONTROL UNIT
22 PROCESSING UNIT
23 STORAGE UNIT
231 PROGRAM
200 EYEBALL
EA EYE AXIS
F FIXATION TARGET
LL ILLUMINATION LIGHT
OL OBSERVATION LIGHT

1. An examination system comprising:
a measurement unit that is movable to change an angle relative to an eyeball of a subject and rotatably moved around an eye axis of the eyeball as a rotation axis;
an illumination unit that is mounted on the measurement unit to irradiate the eyeball;
a first camera unit that is mounted on the measurement unit to image the eyeball; and
a second camera unit that is mounted on the measurement unit to image the eyeball, wherein
the first camera unit and the second camera unit are independently movable on the measurement unit to change angles relative to the eyeball.

2. The examination system according to claim 1, wherein
in a state where the first camera unit and the second camera unit are arranged on opposite sides across the illumination unit,
the measurement unit is moved to change the angle relative to the eyeball, and
the first camera unit and the second camera unit image an anterior segment of the eyeball.

3. The examination system according to claim 1, further comprising
a gonioscope unit that is movable between a position between the eyeball, and the illumination unit and at least one of the first camera unit and the second camera unit, and another position, wherein
in a state where the gonioscope unit is arranged at a position between the eyeball, and the illumination unit and the at least one of the first and second camera units,
the measurement unit rotationally moves around the eye axis of the eyeball as the rotation axis, and
the at least one of the first and second camera units images an angle region of the eyeball.

4. The examination system according to claim 3, further comprising
a reflective optical system unit that is movable between a position between the gonioscope unit, and the illumination unit and the at least one of the first and second camera units, and another position, wherein
in a state where the reflective optical system unit is arranged at a position between the gonioscope unit, and the illumination unit and the at least one of the first and second camera units,
the measurement unit rotationally moves around the eye axis of the eyeball as the rotation axis, and
the at least one of the first and second camera units images the angle region of the eyeball.

5. The examination system according to claim 1, wherein
in a state where the illumination unit is arranged in front of the eyeball, and
where the first camera unit and the second camera unit are arranged on opposite sides across the illumination unit to have angles of the same magnitude relative to the eyeball,
the measurement unit rotationally moves around the eye axis of the eyeball as the rotation axis, and
the first camera unit and the second camera unit image a cornea of the eyeball.

6. The examination system according to claim 1, wherein
in a state where the illumination unit is arranged at a position where the illumination unit has an angle relative to the eyeball,
the measurement unit rotationally moves around the eye axis of the eyeball as the rotation axis, and
the first camera unit and the second camera unit image a crystalline lens of the eyeball.

7. The examination system according to claim 1, wherein
the illumination unit is also independently movable on the measurement unit to change an angle relative to the eyeball, as in the first camera unit and the second camera unit, and
in a state where the illumination unit is arranged at a position where the illumination unit has an angle relative to the eyeball,
the illumination unit, the first camera unit, and the second camera unit move together to change angles relative to the eyeball while maintaining a positional relationship between the illumination unit and the first and second camera units, and
the first camera unit and the second camera unit image a crystalline lens of the eyeball.

8. The examination system according to claim 7, wherein
the measurement unit further rotationally moves around the eye axis of the eyeball as the rotation axis, and
the first camera unit and the second camera unit image the crystalline lens of the eyeball.

9. The examination system according to claim 1, wherein
in a state where the illumination unit, and at least one of the first camera unit and the second camera unit are arranged at positions where the illumination unit and the at least one of the first and second camera units have angles relative to the eyeball,
the measurement unit rotationally moves around the eye axis of the eyeball as the rotation axis, and
the at least one of the first and second camera units images Zinn's zonule of the eyeball.

10. The examination system according to claim 1, wherein
the first camera unit includes an objective zoom optical system,
the second camera unit includes an objective zoom optical system, and
in a state where the illumination unit is arranged at a position where the illumination unit has an angle relative to a line normal to a corneal endothelium as a target to be imaged in the eyeball, where the first camera unit is arranged in front of the corneal endothelium as the target to be imaged, and where the second camera unit is arranged on a side opposite from the illumination unit across the line normal to the corneal endothelium as the target to be imaged, axially symmetrically,
the first camera unit images an entire cornea of the eyeball,
the measurement unit moves to change an angle relative to the eyeball, and the measurement unit rotationally moves around the eye axis of the eyeball as the rotation axis, and
the second camera unit images corneal endothelial cells of the eyeball.

11. The examination system according to claim 1, further comprising
a reflector unit that is movable between a position between the eyeball, and, the illumination unit and at least one of the first camera unit and the second camera unit, and another position, wherein
in a state where the reflector unit is arranged at a position between the eyeball, and the illumination unit and the at least one of the first and second camera units,
each of the illumination unit and the at least one of the first and second camera units is arranged at a position where an angle relative to the eyeball is smaller than an angle of a configuration without the reflector unit.

12. The examination system according to claim 1, wherein
in a state where a fixation target for guiding an orientation of the eyeball is presented to the subject,
the illumination unit illuminates the eyeball, and
at least one of the first camera unit and the second camera unit images the eyeball.

13. The examination system according to claim 1, further comprising
a base unit that is movable forward and backward, leftward and rightward, and vertically, relative to the eyeball, wherein
the measurement unit is mounted on the base unit.

14. The examination system according to claim 1, wherein
the illumination unit irradiates the eyeball with illumination light, and
the illumination light includes slit lamp light.

15. The examination system according to claim 1, further comprising
a processing unit that calculates shapes of regions of the eyeball based on an image of the eyeball imaged by at least one of the first camera unit and the second camera unit, wherein
the processing unit calculates a shape of a region of the regions of the eyeball, positioned in front, and based on ray tracing in consideration of refraction of a ray of light passing through the calculated shape, calculates a shape of a portion positioned in back of the region in front.

16. The examination system according to claim 15, wherein
the processing unit sequentially calculates a shape of an anterior surface of cornea of the eyeball, a shape of a posterior surface of cornea of the eyeball, a shape of an anterior lens capsule of the eyeball, and a shape of a posterior lens capsule of the eyeball to calculate a shape of a crystalline lens of the eyeball.

17. The examination system according to claim 16, wherein
the processing unit calculates an opacity distribution in the crystalline lens of the eyeball.
